(19) United States
(12) Patent Application Publication
(10) Pub. No.: US 2025/0272461 A1
(43) Pub. Date: Aug. 28, 2025

(54) DEVICES AND METHODS FOR MULTIPHASE HYDRATE FRACTION CALCULATION AND ANALYSIS

(71) Applicant: ChampionX LLC, Sugar Land, TX (US)

(72) Inventors: Morteza AMINNAJI, Aberdeen (GB); Alfred HASE, Ellon (GB)

(73) Assignee: ChampionX LLC, Sugar Land, TX (US)

(21) Appl. No.: 19/060,370

(22) Filed: Feb. 21, 2025

Related U.S. Application Data
(60) Provisional application No. 63/559,036, filed on Feb. 28, 2024.

(51) Int. Cl. G06F 30/28; G06F 113/08

(52) U.S. Cl. CPC G06F 30/28; G06F 2113/08

(57) ABSTRACT

Technologies for multiphase hydrate fraction modeling include devices and methods for receiving an initial feed composition of a constant volume cell and a time series of pressure data and temperature data for the cell during a test procedure. The devices and methods include determining a non-equilibrium hydrate fraction for the constant volume cell based on the time series of pressure data and temperature data, predicting one or more phase components of the constant volume cell based on the non-equilibrium hydrate fraction. The devices and methods may include displaying gas composition in the constant volume cell over the time series based on the prediction.

### BACKGROUND

Various methods and algorithms have been developed for thermodynamically calculating multiphase flash, such as vapor-liquid-liquid flash and hydrate flash calculation. For example, the multiphase equilibrium hydrate flash together with stability analysis through Gibbs energy minimization technique is known. This equilibrium multiphase hydrate flash has been implemented in various commercially available programs, such as PVTsim, Multiflash, HydraFlash, and CSMGem. These typical techniques predict the equilibrium hydrate fraction, but do not accurately predict the hydrate fraction, composition, and structure of hydrates formed at various pressure and temperature conditions as a path-dependent during the hydrate formation.

### SUMMARY

According to certain aspects of the disclosure, a method for multiphase hydrate fraction modeling comprises receiving, by a computing device, an initial feed composition of a constant volume cell; receiving, by the computing device, a time series of pressure data and temperature data for the constant volume cell during a test procedure; determining, by the computing device, a non-equilibrium hydrate fraction for the constant volume cell based on the time series of pressure data and temperature data; and predicting, by the computing device, one or more phase components of the constant volume cell based on the non-equilibrium hydrate fraction.

In some embodiments, the initial feed composition is indicative of water volume or mass, liquid hydrocarbon volume or mass, gas volume or mass, and gas composition initially included in the constant volume cell. In some embodiments, the constant volume cell comprises an autoclave. In some embodiments, the constant volume cell comprises an undersea pipeline.

In some embodiments, determining the non-equilibrium hydrate fraction for the constant volume cell based on the time series of pressure data and temperature data comprises, for each time index of the time series: determining whether hydrate formation has occurred based on the pressure data and the temperature data; performing multiphase isothermal hydrate flash by successive substitution to determine a most stable hydrate composition at the time index in response to determining that hydrate formation has occurred; and updating the non-equilibrium hydrate fraction based on the most stable hydrate composition determined by the multiphase isothermal hydrate flash.

In some embodiments, determining whether hydrate formation has occurred comprises determining whether the constant volume cell is in a hydrate stability zone based on the temperature data and the pressure data at the time index; determining phase molar density of non-hydrate phase at the time index by performing an isothermal pressure-temperature (PT) flash in response to determining that the constant volume cell is in the hydrate stability zone; and comparing the phase molar density of the non-hydrate phase to a phase density of the non-hydrate phase at a previous time index to determine whether hydrate formation has occurred. In some embodiments, the most stable hydrate composition is indicative of a hydrate phase and a non-hydrate phase, wherein the hydrate phase is indicative of one or more hydrate structures, and wherein the non-hydrate phase is indicative of a vapor phase or a plurality of liquid phases.

In some embodiments, updating the non-equilibrium hydrate fraction comprises estimating an initial hydrate fraction based on a pressure drop at the time index and a hydration number, wherein the hydration number is based on hydrate composition of the most stable hydrate composition; determining a first molar volume or density of the non-hydrate phase based on the most stable hydrate composition and the initial hydrate fraction; determining a second molar volume or density of the non-hydrate phase based on a pressure-temperature flash for non-hydrate phase composition of the most stable hydrate composition; updating the initial hydrate fraction as a function of the first molar volume or density and the second molar volume or density; and continuing to update the initial hydrate fraction until the first molar volume or density and the second molar volume or density converge to a predetermined percentage error.

In some embodiments, the method further comprises displaying, by the computing device, the non-equilibrium hydrate fraction over the time series. In some embodiments, the method further comprises displaying, by the computing device, gas composition in the constant volume cell over the time series based on predicting the one or more phase components.

According to other aspects, a computing device for multiphase hydrate fraction modeling is disclosed, the computing device comprising a test interface and an analytical engine. The test interface is configured to receive an initial feed composition of a constant volume cell, and to receive a time series of pressure data and temperature data for the constant volume cell during a test procedure. The analytical engine is configured to determine a non-equilibrium hydrate fraction for the constant volume cell based on the time series of pressure data and temperature data, and to predict one or more phase components of the constant volume cell based on the non-equilibrium hydrate fraction.

In some embodiments, the initial feed composition is indicative of water volume or mass, liquid hydrocarbon volume or mass, gas volume or mass, and gas composition initially included in the constant volume cell. In some embodiments, the constant volume cell comprises an autoclave. In some embodiments, the constant volume cell comprises an undersea pipeline.

In some embodiments, to determine the non-equilibrium hydrate fraction for the constant volume cell based on the time series of pressure data and temperature data comprises, for each time index of the time series, to: determine whether hydrate formation has occurred based on the pressure data and the temperature data; perform multiphase isothermal hydrate flash by successive substitution to determine a most stable hydrate composition at the time index in response to a determination that hydrate formation has occurred; and update the non-equilibrium hydrate fraction based on the most stable hydrate composition determined by the multiphase isothermal hydrate flash.

In some embodiments, to determine whether hydrate formation has occurred comprises to: determine whether the constant volume cell is in a hydrate stability zone based on the temperature data and the pressure data at the time index; determine phase molar density of non-hydrate phase at the time index by performance of an isothermal pressure-temperature (PT) flash in response to a determination that the constant volume cell is in the hydrate stability zone; and compare the phase molar density of the non-hydrate phase to a phase density of the non-hydrate phase at a previous time index to determine whether hydrate formation has occurred. In some embodiments, the most stable hydrate composition is indicative of a hydrate phase and a non-hydrate phase, wherein the hydrate phase is indicative of one or more hydrate structures, and wherein the non-hydrate phase is indicative of a vapor phase or a plurality of liquid phases.

In an embodiment, to update the non-equilibrium hydrate fraction comprises to: estimate an initial hydrate fraction based on a pressure drop at the time index and a hydration number, wherein the hydration number is based on hydrate composition of the most stable hydrate composition; determine a first molar volume or density of the non-hydrate phase based on the most stable hydrate composition and the initial hydrate fraction; determine a second molar volume or density of the non-hydrate phase based on a pressure-temperature flash for non-hydrate phase composition of the most stable hydrate composition; update the initial hydrate fraction as a function of the first molar volume or density and the second molar volume or density; and continue to update the initial hydrate fraction until the first molar volume or density and the second molar volume or density converge to a predetermined percentage error.

In some embodiments, the computing device further comprises a user interface to display the non-equilibrium hydrate fraction over the time series. In some embodiments, the computing device further comprises a user interface to display gas composition in the constant volume cell over the time series based on a prediction of the one or more phase components.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a simplified block diagram of at least one embodiment of a system for multiphase hydrate fraction calculation and analysis;

FIG. 2 is a simplified block diagram of an environment that may be established by a computing device of the system of FIG. 1;

FIG. 3 is an example of a flow diagram of at least one embodiment of a method for multiphase hydrate fraction calculation and analysis that may be executed by the computing device of FIGS. 1 and 2;

FIG. 4 is an example of a flow diagram of at least one embodiment of a method for a kinetic path-dependent hydrate flash algorithm that may be executed by the computing device of FIGS. 1 and 2;

FIG. 5 is an example of a flow diagram of at least one embodiment of a method for a non-hydrate phase flash algorithm that may be executed by the computing device of FIGS. 1 and 2;

FIG. 6 is an example of a flow diagram of at least one embodiment of a method for a hydrate phase flash algorithm that may be executed by the computing device of FIGS. 1 and 2;

FIG. 7 is an example of a flow diagram of at least one embodiment of a method for a successive substitution algorithm for energy minimization that may be executed by the computing device of FIGS. 1 and 2; and

FIGS. 8-9 are schematic diagrams illustrating at least one embodiment of a user interface including experimental results that may be generated by the computing device of FIGS. 1 and 2.

### DETAILED DESCRIPTION

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors or processing units (e.g., GPUs, or tensor processing units (TPUs)). A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

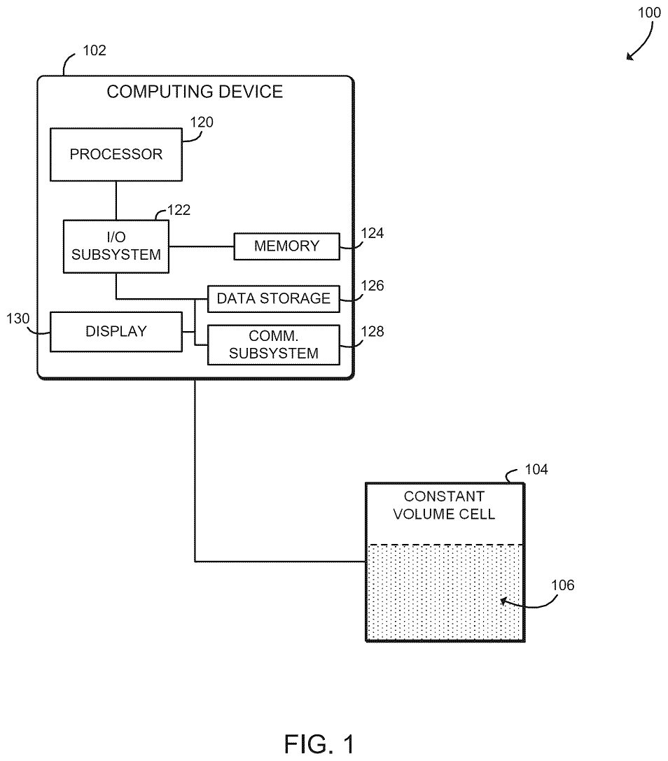

Referring now to FIG. 1, an illustrative system 100 includes a computing device 102 that may be coupled to a constant volume cell 104. In use, as described further below, the computing device receives composition information for an initial feed composition of the cell 104, for example from a user or from one or more sensors or other instruments. The computing device 102 also receives pressure and temperature data for the cell 104 recorded during a test procedure in which temperature of the cell 104 is lowered. Based on that recorded pressure and temperature data, the computing device 102 performs path-dependent kinetic determination of the non-equilibrium hydrate fraction for the cell 104. The computing device 102 may display or otherwise generate composition data, including hydrate and non-hydrate fraction, gas composition, and/or hydrate structure information. Accordingly, the system 100 disclosed herein may use the kinetic multiphase hydrate flash algorithm to dynamically predict hydrate fraction, hydrate structure, and phase composition using data obtained from hydrate growth in the an experiment or in a field application.

Additionally, the system 100 disclosed herein provides analytical insights through enhancing laboratory hydrate studies. The system 100 allows for the kinetic calculation of hydrate fraction, composition, and structure along the entire hydrate growth path, accounting for the interplay of pressure and temperature variations. During the process of hydrate formation, the gas composition undergoes changes, resulting in variations in the composition and potentially even the structure of the hydrates formed. As a result, the composition and structure observed at the final stage of hydrate formation may differ from those observed during the early stages. By providing a robust, pressure-temperature dependent approach, the system 100 serves as a bridge between controlled laboratory experiments and real-world scenarios in the field application.

For example, the system 100 disclosed herein not only enhances understanding of hydrate formation and behavior within a controlled laboratory environment but also offers valuable insights for applications of anti-agglomerants (AAs) in the field. For example, the system 100 not only gives an insight into the performance of AAs as a function of hydrate fraction but also provides a viscosity of hydrate slurry in the presence of AAs (obtained from experiment) as a function of hydrate fraction (predicted from the system 100 as disclosed herein). This viscosity data, correlated with hydrate fraction, can be employed to fine-tune other commercially available software tools for simulating hydrate flow within pipelines.

Furthermore, the system 100 disclosed herein gives an analytical insight into the changes of gas composition during the hydrate growth patch. This composition analysis could be useful for any field process applications that are involved in hydrate formation. Thus, the system 100 disclosed herein opens a new window for safer and more efficient use of AAs where the system 100 is used to predict hydrate formation in subsea pipelines.

Additionally, hydrate formation in production pipelines or porous media may not reach equilibrium conditions within a short period of time. The attainment of equilibrium is influenced by factors such as mixing efficiency and specific conditions, which can significantly prolong the time required to reach equilibrium. Accordingly, a non-equilibrium hydrate fraction calculation as performed by the system 100 may be used for systems that do not reach equilibrium in a short time.

Additionally, while significant progress has been achieved in laboratory methods for evaluating Low Dosage Hydrate Inhibitors (LDHIs), there is still a lack of analytical insights in predicting the hydrate fraction present and multiple hydrate structures during the laboratory experiment. The system 100 disclosed herein invention can greatly assist in the design of enhanced LDHIs.

The computing device 102 may be embodied as any type of device capable of performing the functions described herein. For example, a computing device 102 may be embodied as, without limitation, a workstation, a desktop computer, a laptop computer, a server, a rack-mounted server, a blade server, a network appliance, a web appliance, a tablet computer, a smartphone, a consumer electronic device, a distributed computing system, a multiprocessor system, and/or any other computing device capable of performing the functions described herein. Additionally, in some embodiments, the computing device 102 may be embodied as a “virtual server” formed from multiple computing devices distributed across the network 104 and operating in a public or private cloud. Accordingly, although each computing device 102 is illustrated in FIG. 1 as embodied as a single computing device, it should be appreciated that each computing device 102 may be embodied as multiple devices cooperating together to facilitate the functionality described below. As shown in FIG. 1, the illustrative computing device 102 includes a processor 120, an I/O subsystem 122, memory 124, a data storage device 126, and a communication subsystem 128. Of course, the computing device 102 may include other or additional components, such as those commonly found in a workstation computer (e.g., various input/output devices), in other embodiments. Additionally, in some embodiments, one or more of the illustrative components may be incorporated in, or otherwise form a portion of, another component. For example, the memory 124, or portions thereof, may be incorporated in the processor 120 in some embodiments.

The processor 120 may be embodied as any type of processor or compute engine capable of performing the functions described herein. For example, the processor may be embodied as a single or multi-core processor(s), digital signal processor, microcontroller, or other processor or processing/controlling circuit. Similarly, the memory 124 may be embodied as any type of volatile or non-volatile memory or data storage capable of performing the functions described herein. In operation, the memory 124 may store various data and software used during operation of the computing device 102 such as operating systems, applications, programs, libraries, and drivers. The memory 124 is communicatively coupled to the processor 120 via the I/O subsystem 122, which may be embodied as circuitry and/or components to facilitate input/output operations with the processor 120, the memory 124, and other components of the computing device 102. For example, the I/O subsystem 122 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 122 may form a portion of a system-on-a-chip (SoC) and be incorporated, along with the processor 120, the memory 124, and other components of the computing device 102, on a single integrated circuit chip.

The data storage device 126 may be embodied as any type of device or devices configured for short-term or long-term storage of data such as, for example, memory devices and circuits, memory cards, hard disk drives, solid-state drives, or other data storage devices. The communication subsystem 128 of the computing device 102 may be embodied as any communication circuit, device, or collection thereof, capable of enabling communications between the computing device 102 and other remote devices. The communication subsystem 128 may be configured to use any one or more communication technology (e.g., wireless or wired communications) and associated protocols (e.g., Ethernet, InfiniBand® Bluetooth®, Wi-Fi®, WiMAX, 3G LTE, 5G, etc.) to effect such communication.

As shown, the computing device 102 may include a display 130. The display 130 may be embodied as any type of display capable of displaying digital images or other information, such as a liquid crystal display (LCD), a light emitting diode (LED), a plasma display, a cathode ray tube (CRT), or other type of display device.

Additionally, the computing device 102 may be coupled to the constant volume cell 104. The constant volume cell 104 may be embodied as any closed system with a constant volume. For example, in some embodiments, the constant volume cell 104 may be a test cell such as an autoclave and/or as a production cell such as an undersea pipeline under shut-in condition to be representative of a closed constant volume system. The constant volume cell 104 is a closed system that includes an initial feed 106 material. The feed 106 is a material that may include water (i.e., aqueous liquid), hydrocarbons in a gas phase, hydrocarbons in liquid phase, and/or other materials. The feed 106 has an initial composition, which may be determined in terms of volume or mass.

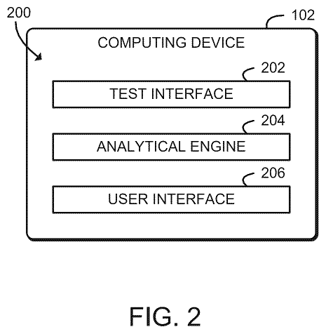

Referring now to FIG. 2, in the illustrative embodiment, the computing device 102 establishes an environment 200 during operation. The illustrative environment 200 includes a test interface 202, an analytical engine 204, and a user interface 206. The various components of the environment 200 may be embodied as hardware, firmware, software, or a combination thereof. As such, in some embodiments, one or more of the components of the environment 200 may be embodied as circuitry or a collection of electrical devices (e.g., test interface circuitry 202, analytical engine circuitry 204, and/or user interface circuitry 206). It should be appreciated that, in such embodiments, one or more of those components may form a portion of the processor 120, the I/O subsystem 122, and/or other components of the computing device 102.

The test interface 202 is configured to receive an initial feed 106 composition of a constant volume cell 104. The test interface 202 is further configured to receive a time series of pressure data and temperature data for the constant volume cell 104 during a test procedure. The initial feed composition may be indicative of water volume or mass, liquid hydrocarbon volume or mass, gas volume or mass, and gas composition initially included in the constant volume cell 104. In some embodiments, the constant volume cell 104 may be autoclave or an undersca pipeline.

The analytical engine 204 is configured to determine a non-equilibrium hydrate fraction for the constant volume cell 104 based on the time series of pressure data and temperature data. The analytical engine 204 is further configured to predict one or more phase components of the constant volume cell 104 based on the non-equilibrium hydrate fraction.

In some embodiments, determining the non-equilibrium hydrate fraction for the constant volume cell based on the time series of pressure data and temperature data includes, for each time index of the time series, determining whether hydrate formation has occurred based on the pressure data and the temperature data, performing multiphase isothermal hydrate flash by successive substitution to determine a most stable hydrate composition at the time index if hydrate formation has occurred, and updating the non-equilibrium hydrate fraction based on the most stable hydrate composition determined by the multiphase isothermal hydrate flash. In some embodiments, determining whether hydrate formation has occurred includes determining whether the constant volume cell is in a hydrate stability zone based on the temperature data and the pressure data at the time index, and if so, determining phase molar density of non-hydrate phase at the time index by performance of an isothermal pressure-temperature (PT) flash, and comparing the phase molar density of the non-hydrate phase to a phase density of the non-hydrate phase at a previous time index. The most stable hydrate composition may be indicative of a hydrate phase and a non-hydrate phase. The hydrate phase is indicative of one or more hydrate structures, and the non-hydrate phase is indicative of a vapor phase or a plurality of liquid phases. Updating the non-equilibrium hydrate fraction may include estimating an initial hydrate fraction based on a pressure drop at the time index and a hydration number, determining a molar volume or density of the non-hydrate phase based on the most stable hydrate composition and the initial hydrate fraction; determining another molar volume or density of the non-hydrate phase based on a pressure-temperature flash for non-hydrate phase composition of the most stable hydrate composition, updating the initial hydrate fraction as a function of both of the molar volumes or densities, and continuing to update the initial hydrate fraction until the molar volumes or densities converge to a predetermined percentage error. The hydration number is based on hydrate composition of the most stable hydrate composition.

The user interface 206 is configured to display the non-equilibrium hydrate fraction over the time series. The user interface 206 may be further configured to display gas composition in the constant volume cell over the time series based on a prediction of the one or more phase components.

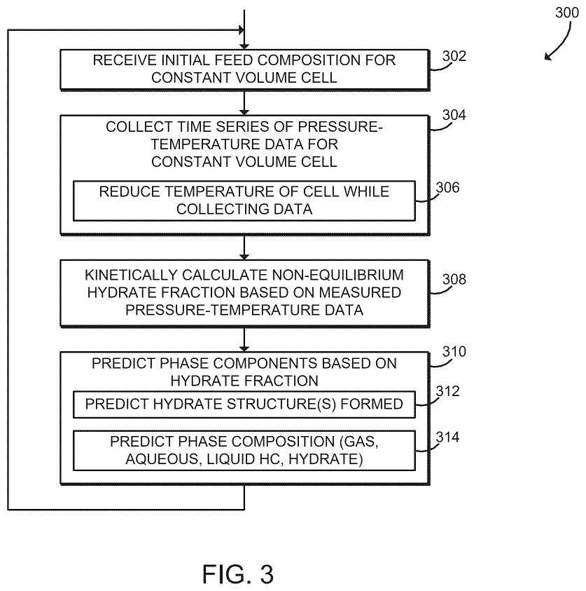

Referring now to FIG. 3, in use, the computing device 102 may execute a method 300 for multiphase hydrate fraction calculation and analysis. It should be appreciated that, in some embodiments, the operations of the method 300 may be performed by one or more components of the environment 200 of the computing device 102 as shown in FIG. 2. The method 300 begins with block 302, in which the computing device 102 receives an initial feed 106 composition for the constant volume cell 104. For example, the constant volume cell 104 may be filled with aqueous liquid phase (and/or liquid hydrocarbon) and then pressurized with gas. The computing device 102 may receive data indicative of the mass and/or volume of the various phases included in the initial feed 106. For example, the initial feed 106 composition may indicate aqueous phase mass or volume, liquid hydrocarbon mass or volume, and/or gas mass or volume (including pressure and temperature). The initial feed composition 106 may also include composition data for the gas phase, for example molar percentages of gas components. The initial feed composition 106 may be provided by a user, received from one or more sensors, another computing device, or otherwise received by the computing device 102.

In block 304, the computing device 102 collects a time series of pressure-temperature data for the constant volume cell 104. The computing device 102 may receive the pressure-temperature data, for example, directly from one or more sensors, from another computer device, as a data file, or otherwise receive data indicative of pressure and temperature in the constant volume cell 104 over time. In particular, the pressure-temperature data may be collected during an isochoric procedure in the constant volume cell 104. In block 306, temperature of the constant volume cell 104 is reduced while the pressure-temperature data is collected. As the temperature is reduced, the system is brought into the hydrate stability zone (HSZ), which may induce hydrate formation.

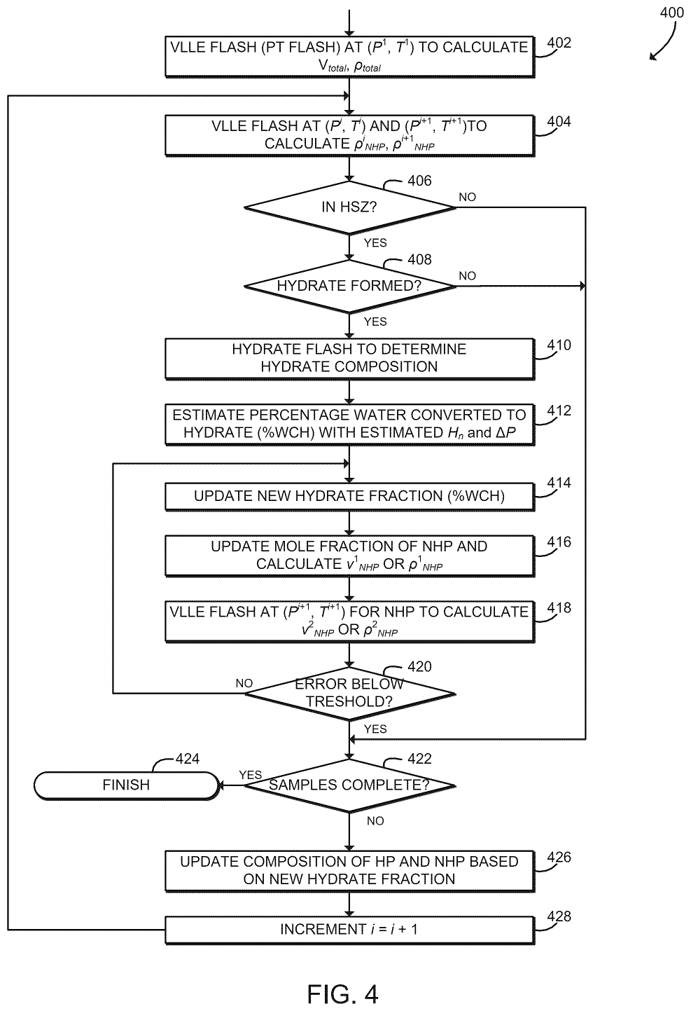

In block 308, the computing device 102 kinetically calculates the non-equilibrium hydrate fraction based on the measured pressure-temperature data. The hydrate fraction and composition depend on the hydrate growth path. Therefore, a non-equilibrium kinetic hydrate flash algorithm is required to kinetically calculate hydrate fraction as pressure-temperature path dependent. In the illustrative embodiment, a multiphase isothermal flash algorithm (Vapor-Liquid-Liquid-Hydrate structure I-Hydrate structure II) and a kinetic hydrate flash algorithm arc used as a path-dependent approach for predicting hydrate composition and fraction throughout the path during the procedure. Illustratively, a modified van der Waals and Plattecuw method is used for the sI and sII hydrate phases, and the s-CPA (cubic plus association) equation of state as an association term with the Soave-Redlich-Kwong (SRK) equation of state as a non-associated part for the non-hydrate phase is used. Stability analysis and the Hessian matrix approach are applied for Gibbs minimization to determine the most stable hydrate structure/composition. The determination of the non-equilibrium hydrate flash algorithm is described further below in connection with FIGS. 4-7.

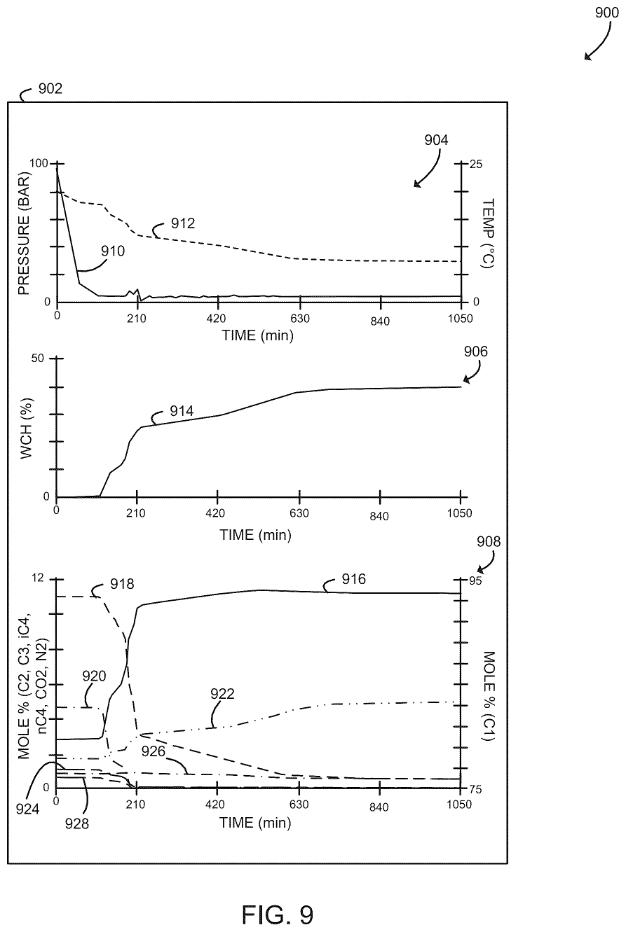

In block 310, the computing device 102 predicts one or more phase components based on the hydrate fraction. In some embodiments, the computing device 102 may provide a user interface including data visualization that represents the predicted phase components. Additionally or alternatively, the computing device 102 may output detailed results predicting phase composition over time that may be further processed or analyzed. One potential embodiment of a user interface for performing an analysis and visualizing results is shown in FIGS. 8-9 and described further below. In block 312, the computing device 102 may predict one or more hydrate structures formed during the experiment. For example, the computing device 102 may predict the molar composition of hydrates in the constant volume cell 104, along with predicting different hydrate structures (e.g., sl and/or sII) that may form simultaneously during the experiment. In block 314, the computing device 102 may predict the phase composition for the constant volume cell 104 during the procedure. For example, the computing device 102 may predict molar percentages of each phase in the constant volume cell 104 (e.g., aqueous phase, liquid hydrocarbon phase, gas phase, and/or hydrate phases sI, sII). The computing device 102 may also predict composition of the gas phase, including molar percentages for each component gas of the gas phase. After predicting the phase composition, the method 300 loops back to block 302, in which the computing device 102 may continue to process temperature and pressure data.

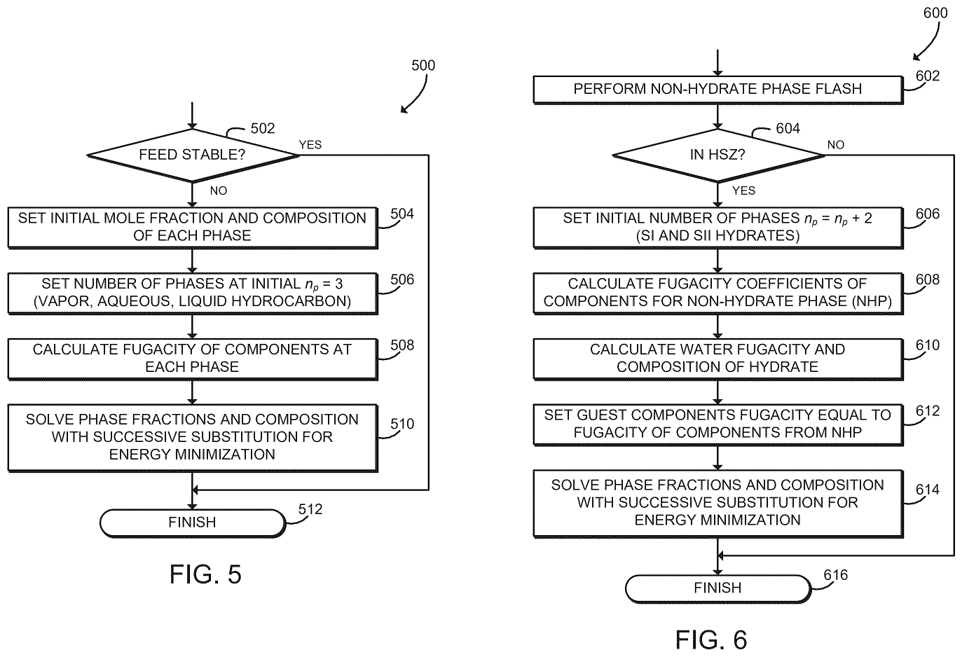

Referring now to FIG. 4, in use, the computing device 102 may execute a method 400 for a kinetic path-dependent hydrate flash algorithm. The method 400 may be executed, for example, in connection with the method 300 of FIG. 3. It should be appreciated that, in some embodiments, the operations of the method 400 may be performed by one or more components of the environment 200 of the computing device 102 as shown in FIG. 2. The method 400 begins with block 402, in which the computing device 102 performs an isothermal pressure-temperature (PT) flash at the initial data point (i.e., P¹, T¹) for the initial feed composition to determine the vapor-liquid-liquid equilibrium (VLLE) and subsequently to calculate the total density or total molar volume of the system (e.g., ρₜₒₜₐₗ, Vₜₒₜₐₗ). It is assumed that the hydrate fraction is zero at the initial entry point of the method 400, and that gas and liquid hydrocarbon phase (if present) compositions are known. It is further assumed that the amounts of water, gas, and liquid hydrocarbon are known, cither in terms of mass or volume. One potential embodiment of a method for a general flash algorithm is described further below in connection with FIGS. 5-7.

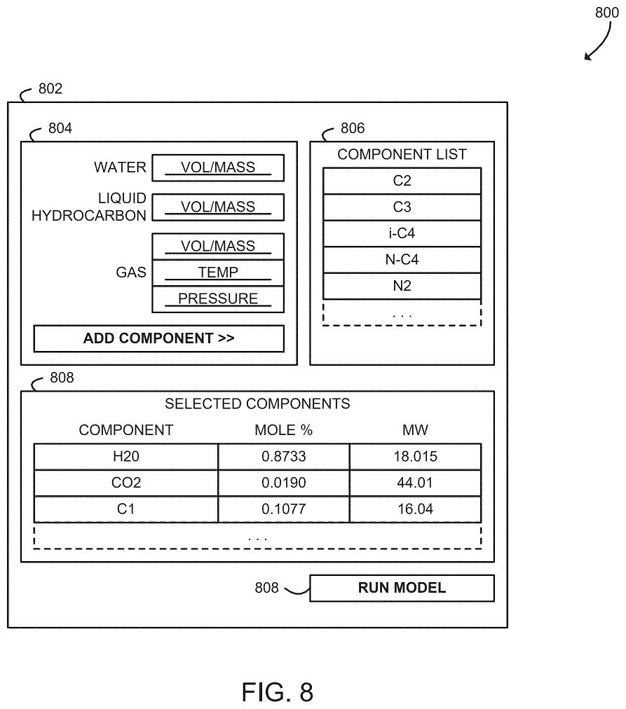

In block 404, the computing device 102 performs another isothermal PT flash at the subsequent data point (Pⁱ⁺¹, Tⁱ⁺¹) to assess whether hydrate formation is occurred between the previous data point (Pⁱ, Tⁱ) and subsequent data point (Pⁱ⁺¹, Tⁱ⁺¹). In some embodiments, the computing device 102 may perform a non-hydrate phase (NHP) flash algorithm to determine VLLE at both (Pⁱ, Tⁱ) and (Pⁱ⁺¹, Tⁱ⁺¹). One potential embodiment of an NHP flash algorithm is described below in connection with FIGS. 5 and 7. In block 406, the computing device 102 determines whether the constant volume cell 104 is in the hydrate stability zone (HSZ) at data point i+1. For example, the computing device may determine whether the measured temperature Tⁱ⁺¹ is less than an equilibrium temperature T^{eq} at the measured pressure Pⁱ⁺¹. If not, the method 400 skips ahead to block 422, described below. If so, the method 400 proceeds to block 408, in which the computing device 102 determines whether hydrate has formed during the current time interval. The computing device 102 may determine whether density of the non-hydrate phase decreased, for example by determining whether molar density of the non-hydrate phase at the previous time ρ_{NHP}ⁱ is greater than molar density of the non-hydrate phase at the subsequent time ρ_{NHP}ⁱ⁺¹. If not, the method 400 skips ahead to block 422, described below. If so, the method 400 proceeds to block 410.

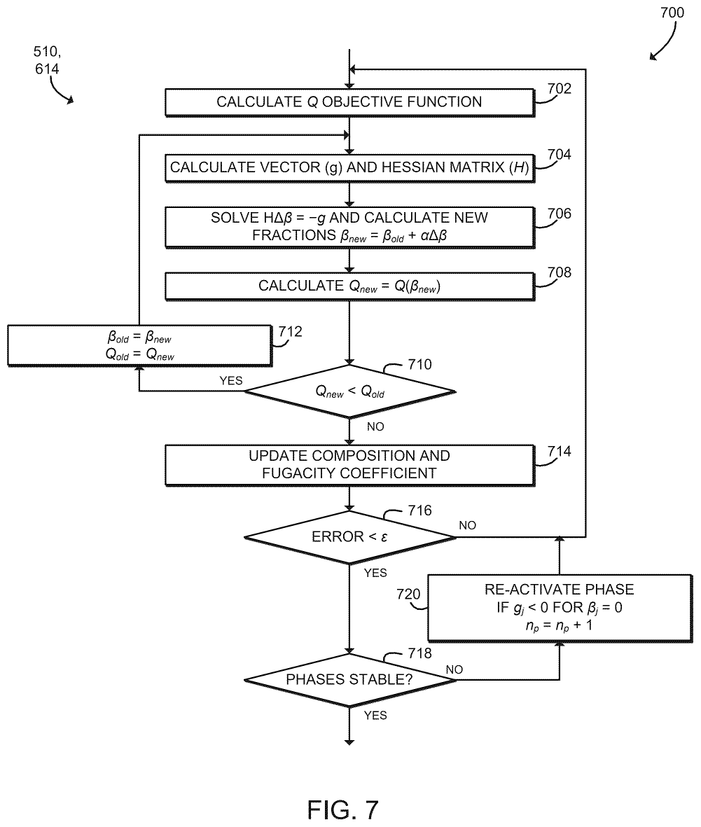

In block 410, after determining that hydrate formation has occurred, the computing device 102 performs multiphase isothermal hydrate Flash (Vapor-Liquid-Liquid-Hydrate) through a successive substitution algorithm developed by Michelsen for Gibbs energy minimization. One potential embodiment of the hydrate flash algorithm is described further below in connection with FIGS. 6 and 7. As a result of the hydrate flash, the computing device 102 determines the composition and the most stable hydrate structure at the current pressure and temperature. Any potential hydrate structure (e.g., sI and sII) can be included as an independent phase in the multiphase isothermal hydrate Flash. This allows for the identification of simultaneous formation of different stable hydrate structures if present.

Once the most stable hydrate composition is found, the hydrate fraction formed from data point i to i+1 may be calculated by updating hydrate mole fraction to adjust the molar volume (or density) of the non-hydrate phase (NHP). In order to accurately calculate the amount of hydrate formed from data point i to i+1, in block 412 an initial hydrate fraction is estimated using Equation 1, below, by considering pressure drop (ΔP=Pⁱ-Pⁱ⁺¹) and hydration number (Hₙ) which is calculated by the hydrate composition in the multiphase isothermal hydrate Flash.

$$WCH \% = {H}_{n} \frac{Δ P}{{p}^{i}} \frac{1 - {f}_{w}}{{f}_{w}} \qquad ( 1 )$$

where WCH % is the percentage of water converted to hydrate and f_{w} is the water mole fraction.

In block 414, the computing device 102 updates a new hydrate fraction. Initially, the new hydrate fraction may be set to the initial, estimated value determined in block 412. In subsequent iterations, the hydrate fraction may be updated based on updated calculations as described below. In block 416, the computing device 102 determines the molar volume or density of the non-hydrate phase (NHP) using Equation 2, below, by considering the volume occupied and the number of moles used by hydrates according to the hydrate composition and estimated hydrate fraction. The volume occupied by hydrates can be estimated using the density of empty clathrate for the potential hydrate structure (e.g., sI and sII) obtained through molecular dynamics (MD) simulations.

$${ν}_{NHP}^{1} = \frac{{V}_{total} - {V}_{H}}{{n}_{total} - {n}_{HP}} \qquad ( 2 )$$

In block 418, the computing device 102 updates the mole fraction of NHP based on the hydrate composition and estimated hydrate fraction. Therefore, a new molar volume or density (v_{NHP}²) can be calculated using PT flash (Vapor-Liquid-Liquid) for only NHP with the new composition.

In block 420, the computing device 102 determines whether an error based on the calculated molar volumes or densities is below a predetermined threshold. For example, the estimated or previous hydrate fraction formed from data points i to i+1 may be verified by calculating the relative error between the two calculated molar volumes or densities

$$( \frac{{ν}_{NHP}^{1} - {ν}_{NHP}^{2}}{{ν}_{NHP}^{1}} ) .$$

If this error exceeds a certain threshold, the hydrate fraction needs to be updated within the iteration loop. In some embodiments, a 1% error threshold may be used for rapid convergence of the algorithm. If the error exceeds the threshold, the method 400 loops back to block 414 to update the hydrate fraction and recalculate molar volumes or densities. A new hydrate fraction (e.g., percentage of water converted to hydrate) can be updated using Equation 3, below. In some embodiments, to converge the algorithm, the change and update to the hydrate fraction may be limited to a maximum of 5% for each iteration.

$$WCH {\%}^{new} = WCH {\%}^{old} ( \frac{{ν}_{NHP}^{1}}{{ν}_{NHP}^{2}} ) \qquad ( 3 )$$

Once the convergence is occurred, the same procedure is carried out for the other remaining experimental data points through the hydrate growth path. Referring again to block 420, if the error is below the threshold, the method 400 proceeds to block 422. In block 422, the computing device 102 determines whether additional data points (Pⁱ, Tⁱ) exist. If not, the method branches to block 424 and is completed. If so, the method 400 advances to block 426, in which the computing device 102 updates the composition of the hydrate phase and the non-hydrate phase based on the new hydrate fraction determined as described above. In block 428, the computing device 102 increments i=i+1 in order to process the next recorded data point. After incrementing i, the method 400 loops back to block 404 to continue performing the kinetic flash algorithm.

Referring now to FIG. 5, in use, the computing device 102 may execute a method 500 for a non-hydrate phase flash algorithm. The method 500 may be executed, for example, in connection with the VLLE flash described in connection with blocks 402, 404, 418 of the method 400, described above. It should be appreciated that, in some embodiments, the operations of the method 500 may be performed by one or more components of the environment 200 of the computing device 102 as shown in FIG. 2. The method 500 begins with block 502, in which the computing device 102 determines whether the feed is stable. If the feed is stable, the method 500 branches ahead to block 512, in which the method 500 is completed. The phase composition is not updated. If the feed is not stable, the method 500 advances to block 504.

In block 504, the computing device 102 sets an initial mole fraction and composition of each phase. In block 506, the computing device 102 sets the initial number of phases at the initial time to three (nₚ=3), for vapor (V), aqueous (A), and liquid hydrocarbon (H) phases. In block 508, the computing device 102 calculates the fugacity of components at each phase. In block 510, the computing device 102 solves for phase fractions and phase composition using a successive substitution algorithm for Gibbs free energy minimization. One potential embodiment of a method for solving the phase fractions is described further below in connection with FIG. 7. After solving the phase fractions and phase composition, the method 500 advances to block 512, in which the method 500 is completed.

Referring now to FIG. 6, in use, the computing device 102 may execute a method 600 for a hydrate phase flash algorithm. The method 600 may be executed, for example, in connection with the hydrate flash performed in connection with block 410 of the method 400, described above. It should be appreciated that, in some embodiments, the operations of the method 600 may be performed by one or more components of the environment 200 of the computing device 102 as shown in FIG. 2. The method 600 begins with block 602, in which the computing device 102 performs a non-hydrate phase flash. The computing device 102 may, for example, perform the method 500 described above in connection with FIG. 5. In block 604, the computing device 102 determines whether current conditions of the constant volume cell 104 (e.g., Pⁱ, Tⁱ or other data point) are within the hydrate stability zone (HSZ). The computing device 102 may, for example, determine whether Tⁱ<T^{eq} at Pⁱ or otherwise determine whether hydrates may form at the current conditions. If not, the method 600 branches ahead to block 616, in which the method 600 is completed. If the conditions are within the HSZ, the method 600 advances to block 606.

In block 606 the computing device 102 increases the initial number of phases by the number of hydrate structures. For example, for two hydrate structures sI and sII, nₚ=nₚ+2. In block 608, the computing device 102 calculates the fugacity coefficients of components for each non-hydrate phase. In block 610, the computing device 102 calculates water fugacity and composition of hydrate. In block 612, the computing device 102 sets guest components fugacity equal to the fugacity of components from the non-hydrate phase. In block 614, the computing device 102 solves for phase fractions and composition of each phase using a successive substitution algorithm for Gibbs free energy minimization. One potential embodiment of a method for solving the phase fractions is described further below in connection with FIG. 7. After solving the phase fractions and phase composition, the method 600 advances to block 616, in which the method 600 is completed.

Referring now to FIG. 7, in use, the computing device 102 may execute a method 700 for a successive substitution algorithm for energy minimization. The method 700 may be executed, for example, in connection with solving phase fractions and phase composition as described above in connection with block 510 of the method 500 and block 614 of the method 600. It should be appreciated that, in some embodiments, the operations of the method 700 may be performed by one or more components of the environment 200 of the computing device 102 as shown in FIG. 2. The method 700 begins with block 702, in which the computing device 102 calculates an objective function Q.

Although different methods and algorithms have been developed for multiphase flash, as described above, the successive substitution algorithm developed by Michelsen for Gibbs energy minimization is used for both hydrate and non-hydrate multiphase flash. The equilibrium state is subjected to minimize the Gibbs free energy at global state that can be express by the Equations 4 and 5, below.

$$\frac{G}{RT} = {\sum }_{i}^{{n}_{c}} {μ}_{i}^{0} {z}_{i} ( \frac{{μ}_{i}^{0}}{RT} + ln ( {z}_{i} P ) ) - {\sum }_{i}^{{n}_{c}} {z}_{i} ln ( {E}_{i} ) \qquad ( 4 )$$

$${E}_{i} = {\sum }_{k = 1}^{{n}_{p}} \frac{{β}_{k}}{{∅}_{ik}} \qquad ( 5 )$$

The Gibbs free energy minimization at global state is subjected to minimize the objective function, Q(β), with the constraints of

$$\frac{\partial Q}{\partial {β}_{j}} = 0 , {β}_{j} \geq 0 or \frac{\partial Q}{\partial {β}_{j}} > 0 , {β}_{j} = 0.$$

Therefore, gradient of objective function (gᵢ) and Hessian matrix (Hⱼₖ) can be defined accordingly, as shown below in Equations 6-8.

$$Q ( β ) = {\sum }_{j = 1}^{{n}_{p}} {β}_{j} - {\sum }_{i}^{{n}_{c}} {z}_{i} ln ( {E}_{i} ) \qquad ( 6 )$$

$${g}_{j} = \frac{\partial Q}{\partial {β}_{j}} = 1 - {\sum }_{i}^{{n}_{c}} \frac{{z}_{i}}{{E}_{i}} \frac{1}{{∅}_{ij}} \qquad ( 7 )$$

$${H}_{j k} = \frac{\partial {g}_{j}}{\partial {β}_{k}} = {\sum }_{i = 1}^{{n}_{c}} \frac{{z}_{i}}{{E}_{i}^{2} {∅}_{ij} {∅}_{ik}} \qquad ( 8 )$$

In block 704, the computing device 102 calculates the vector (g) and the Hessian matrix (H). In block 706, the computing device 102 calculates a new phase fraction by solving HΔβ+g=0 to minimize the objective function. The phase fractions including the hydrate phase is determined by β_{new}=β_{old}+αΔB. If any new fraction is negative, α<1 may be picked such that the fraction becomes zero. The Hessian matrix is positive definite where the number of phases is not greater than the number of components which guarantees a unique solution (i.e., Newton's method, coupled with a line search, proves to be an efficient and highly solution procedure). In block 708, the computing device 102 calculates Q_{new}=Q(β_{new}). In block 710, the computing device 102 determines whether Q_{new}<Q_{old}. If so, the method branches to block 712, in which values for β and Q are updated (i.e., β_{old}=β_{new} and Q_{old}=Q_{new}). After updating β and Q, the method 700 loops back to block 704 to continue minimizing the objective function Q. Referring again to block 710, once Q is minimized, the method 700 advances to block 714.

In block 714, the computing device 102 updates composition and fugacity coefficients for each phase. The mole fractions in each phase for the final solution can be determined by Equation 9, below.

$${y}_{jj} = \frac{{z}_{i}}{{E}_{i}} \frac{1}{{∅}_{ij}} \qquad ( 9 )$$

Since the fugacity components of each phase should be equal at equilibrium, the flash calculation of the method 700 continues until it reaches a specific minimum error (e.g., ε=10⁻¹⁰), as defined in Equation 10, below. In equation 10, fᵢᵣ is defined as the fugacity of any active phase. Accordingly, in block 716, the computing device 102 determines whether error is less than the predetermined minimum error ε. If not, the method 700 loops back to block 702 to continue minimizing the objective function Q. If the error is below E, the method 700 advances to block 718, in which the computing device 102 determines whether the phases are stable. If so, the method 700 is completed. If not, the method 700 branches to block 720, in which the computing device 102 reactivates a phase if gⱼ<0 for βⱼ=0, and increments the number of phases nₚ=nₚ+1. After reactivating a phase, the method 700 loops back to block 702 to continue minimizing the objective function Q.

$$Error = {\sum }_{j}^{{n}_{p}^{active}} {\sum }_{i}^{{n}_{c}} \frac{\vert {f}_{ij} - {f}_{ir} \vert}{{f}_{ir}} \qquad ( 10 )$$

The computing device 102 may use different thermodynamic models for the vapor, liquid, and aqueous phases as compared to the hydrate phase. In particular, the Cubic Plus Association (CPA) equation of state (EoS) combined with the Soave-Redlich-Kwong (SRK) equation of state is used to describe the non-hydrate phase. Considering the association part is crucial for hydrate thermodynamic modelling due to hydrogen bonding where water is present. The equation of state for associating fluids is shown below as Equation 11. In Equation 11,

$$\frac{RT}{v - b} - \frac{a ( T )}{v ( v + b )}$$

is the associating part, and

$$\frac{1}{2} \frac{RT}{v} ( 1 + ρ \frac{\partial ln ( g )}{\partial ρ} ) {\sum }_{i}^{N} {y}_{i} {\sum }_{{A}_{i}} ( 1 - {X}^{{A}_{i}} )$$

is the non-associating part.

$$P = \frac{RT}{v - b} - \frac{a ( T )}{v ( v + b )} - \frac{1}{2} \frac{RT}{v} ( 1 + ρ \frac{\partial ln ( g )}{\partial ρ} ) {\sum }_{i}^{N} {y}_{i} {\sum }_{{A}_{i}} ( 1 - {X}^{{A}_{i}} ) \qquad ( 11 )$$

In this disclosure, the SRK ESo is used to describe the non-association cubic part to account van der Waals forces for physical interaction. As disclosed, a(T) and b are attractive and co-volume parameters which may be calculated using Equations 12-17, below.

$$a ( T ) = {\sum }_{i}^{N} {\sum }_{j}^{N} {x}_{i} {x}_{j} {a}_{ij} \qquad ( 12 )$$

$${a}_{ij} = \sqrt{{a}_{i} {a}_{j}} ( 1 - {k}_{ij} ) \qquad ( 13 )$$

$${a}_{i} ( T ) = {Ω}_{a} \frac{{R}^{2} {T}_{c , i}^{2}}{{P}_{c , i}^{2}} {( 1 + {m}_{i} ( 1 - \sqrt{\frac{T}{{T}_{C , i}}} ) )}^{2} \qquad ( 14 )$$

$$b = {\sum }_{i}^{N} {x}_{i} {b}_{i} \qquad ( 15 )$$

$${b}_{i} = {Ω}_{b} \frac{{RT}_{c , i}}{{P}_{c , i}} \qquad ( 16 )$$

$${m}_{i} = 0.48508 + 1.55171 {ω}_{i} - 0.15613 {ω}_{i}^{2} \qquad ( 17 )$$

The binary interaction parameters (kᵢⱼ) may be estimated through the functional group contribution method developed by Jaubert et al., and described in Equation 18, below.

$$( 18 )$$

$${k}_{i j} ( T ) = \frac{\begin{matrix}( - \frac{1}{2} [ {\sum }_{K = 1}^{{N}_{g}} {\sum }_{l = 1}^{{N}_{g}} ( {α}_{i k} - {α}_{j k} ) ( {α}_{il} - {α}_{jl} ) {{A}_{kl} ( \frac{298.15}{T} )}^{( \frac{{B}_{kl}}{{A}_{kl}} - 1 )} ] - \\ {( \frac{\sqrt{{a}_{i} ( T )}}{{b}_{i}} - \frac{\sqrt{{a}_{j} ( T )}}{{b}_{j}} )}^{2} )\end{matrix}}{2 \frac{\sqrt{{a}_{i} ( T ) j ( T )}}{{b}_{i} {b}_{j}}}$$

where αᵢₖ is the fraction occupied by group k in the molecule i and Ng is the number of functional groups. The constant parameters of Aₖₗ and Bₖₗ may be calculated for the Peng-Robinson equation of state, however, they can also be used for SRK EoS.

In the association part of CPA EoS, g represents the radial distribution function, which can be expressed in a simplified form as follows in Equation 19:

$$g = \frac{1}{1 - 1.9 η} , η = \frac{b ρ}{4} \qquad ( 19 )$$

X^{A}^{ᵢ} is the fraction of component i molecules where the A sites remain unassociated (i.e., free to form hydrogen bonds) and can be represent by Equation 20. X^{A}^{ᵢ} is a function of density (ρ), the fractions of all other types of association sites B (X^{B}^{ⱼ}), and the association strength between an A site on a molecule i and a B site on a molecule of component j (δ^{A}^{ᵢ}^{B}^{ⱼ}). The association strengths can be calculated using Equation 21, below, where ε^{A}^{ᵢ}^{B}^{ⱼ} and β^{A}^{ᵢ}^{B}^{ⱼ} are the association energy and association volume for the hydrogen bond between sites A and B in molecules i and j respectively. These parameters, which are determined by fitting the experimental data, have been reported for various molecules. In this disclosure, the water molecule is considered the only self-associating component, while the other component (e.g., CO₂) can form associations with other molecules (e.g., water) but does not self-associate or form cross associations with each other. Of course, in other embodiments, different approaches may be used in terms of self- and cross-association of molecules.

$${X}^{{A}_{i}} = \frac{1}{1 + ρ {\sum }_{j} {x}_{j} {\sum }_{{B}_{j}} {X}^{{B}_{j}} {δ}^{{A}_{i} {B}_{j}}} \qquad ( 20 )$$

$${δ}^{{A}_{i} {B}_{j}} = g ( exp ( \frac{{ε}^{{A}_{i} {B}_{j}}}{RT} ) - 1 ) {β}^{{A}_{i} {B}_{j}} {b}_{ij} \qquad ( 21 )$$

Finally, after solving the CPA-SRK EOS, the fugacity coefficient of the component i(Øᵢ) can be calculated using the following Equations 22-24:

$$ln {∅}_{i} = ln {∅}_{i}^{phys} + ln {∅}_{i}^{assoc} \qquad ( 22 )$$

$$ln {∅}_{i}^{phys} = \frac{{b}_{i}}{b} ( Z - 1 ) - ln ( Z - B ) + \frac{A}{B} ( \frac{2 {\sum }_{j}^{n} {x}_{j} {a}_{i j}}{a} - \frac{{b}_{i}}{b} ) ln ( \frac{Z}{Z + B} ) , \\ A = \frac{a P}{{R}^{2} {T}^{2}} , B = \frac{b P}{R T} \qquad ( 23 )$$

$$ln {∅}_{i}^{assoc} = {\sum }_{{A}_{i}} ln {X}_{{A}_{i}} + \frac{{B}_{i}}{8 g Z} \frac{d g}{d η} {\sum }_{k = 1}^{c} {x}_{k} {\sum }_{{A}_{k}} ( {X}_{{A}_{k}} - 1 ) \qquad ( 24 )$$

For the hydrate phase, a modified version of van der Waals and Platteeuw for solid solution developed by Parrish and Prausnitz is used to calculate the fugacity of water in the hydrate phase as a non-ideal phase. The following Equation 25 can be used to calculate the fugacity of water in the hydrate phase (f_{w}^{H}), where f_{w}^{β} is the fugacity of water in the hypothetical empty hydrate lattices.

$${f}_{w}^{H} = {f}_{w}^{β} exp ( - \frac{Δ {μ}_{w}^{β - H}}{RT} ) \qquad ( 25 )$$

In Equation 25, Δμ_{w}^{β-H} is the chemical potential difference between the hypothetical empty hydrate lattices and hydrate that can be calculated using the following equation 26:

$$Δ {μ}_{w}^{β - H} = {μ}_{w}^{β} - {μ}_{w}^{H} = RT {\sum }_{m}^{{n}_{c a v}} {v}_{m} ln ( 1 + {\sum }_{j}^{{n}_{h}} {C}_{mj} {f}_{j} ) \qquad ( 26 )$$

where vₘ is the number of cavities of type m per water molecule in hydrate and fⱼ is the fugacity of guest component j which is assumed equal to the fugacity of component j in non-hydrate phase that are calculated using the CPA-SRK EoS. The Langmuir constant (Cₘⱼ) which is the gas-water molecules interactions in the hydrate cavities can be calculated by Equations 27-29, below. The spherically symmetric cell potential in the cavity (Wₘⱼ(r)) consider the intermolecular interaction between the guest and host molecules and can be expressed as a function of Kihara parameters (ε_{c}, σ_{c}, and a_{c}).

$${C}_{mj} ( T ) = \frac{4 π}{{K}_{B} T} {\int }_{0}^{∞} exp ( - \frac{{W}_{mj} ( r )}{{K}_{B} T} ) {r}^{2} dr \qquad ( 27 )$$

$${W}_{mj} ( r ) = 2 {z}_{w} {ε}_{c} [ \frac{{σ}_{c}^{12}}{{R}_{c}^{1 {1}_{r}}} ( {δ}^{10} + \frac{{a}_{c}}{{R}_{c}} {δ}^{11} ) - \frac{{σ}_{c}^{6}}{{R}_{c}^{5} r} ( {δ}^{4} + \frac{{a}_{c}}{{R}_{c}} {δ}^{5} ) ] \qquad ( 28 )$$

$${δ}^{N} = \frac{1}{N} [ {( 1 - \frac{r}{{R}_{c}} - \frac{{a}_{c}}{{R}_{c}} )}^{- N} - {( 1 + \frac{r}{{R}_{c}} - \frac{{a}_{c}}{{R}_{c}} )}^{- N} ] N = 4 , 5 , 10 , 11 \qquad ( 29 )$$

An analogous approach is used to calculate the fugacity of water in the hypothetical empty hydrate lattice as expressed in Equation 30 where f_{w}^{L} is the fugacity of pure liquid water and Δμ_{w}^{β-L} is the difference between chemical potential of empty hydrate lattice and pure liquid water. Δμ_{w}^{β-L} can be calculated through Equations 31-33, below. The reference values for the thermodynamic properties (Δμ_{w}⁰, Δh_{w}⁰, and Δv_{w}) in the hydrate structure I and structure II have been reported. The molar heat capacity different (ΔC_{pw}^{β-L}) can be also calculated using Equation 33. The other properties such as cage radius and coordination number for hydrate structure I and structure II have been reported.

$${f}_{w}^{β} = {f}_{w}^{L} exp ( - \frac{Δ {μ}_{w}^{β - L}}{RT} ) \qquad ( 30 )$$

$$\frac{Δ {μ}_{w}^{β - L}}{RT} = \frac{Δ {μ}_{w}^{0}}{{RT}_{0}} - {\int }_{{T}_{0}}^{T} \frac{Δ {h}_{w}^{β - L}}{{RT}^{2}} d T + {\int }_{{P}_{0}}^{P} \frac{Δ {v}_{w}^{β - L}}{RT} dP \qquad ( 31 )$$

$$Δ {h}_{w}^{β - L} = Δ {h}_{w}^{0} + {\int }_{{T}_{0}}^{T} Δ {C}_{pw}^{β - L} ( T ) dT \qquad ( 32 )$$

$$Δ {C}_{pw}^{β - L} ( T ) = Δ {C}_{pw}^{β - L} ( {T}_{0} ) + Δ {b}^{β - L} ( T - {T}_{0} ) \qquad ( 33 )$$

Referring now to FIG. 8, diagram 800 illustrates an example of a user interface 802 that may be provided by the computing device 102 (or another device in communication with the computing device 102). The user interface 802 may be embodied as an interactive user application, for example developed using C++ OOP (Object-Oriented Programming) or any other appropriate development environment. The illustrative interface 802 includes input panels 804, 806, 808, which allow the user to input or otherwise manage the total feed composition and/or gas composition for the constant volume cell 104. The interface 802 also includes a control 808 that allows a user to initiate modeling. When initiating modeling, the computing device 102 may receive pressure-temperature data, which may be entered manually by the user, read from one or more data files, received from one or more sensors, or otherwise received by the computing device 102.

Referring now to FIG. 9, diagram 900 illustrates another example of a user interface 902 that may be provided by the computing device 102. The interface 902 may be provided by the computing device 102 during and/or after modeling the system as described above. Illustratively, the interface 902 includes a pressure-temperature chart 904, a hydrate fraction chart 906, and a gas composition chart 908. The pressure-temperature chart 904 includes a temperature curve 910 and a pressure curve 912 representing the pressure-temperature data for the constant volume cell 104. The hydrate fraction chart 906 includes a WCH (%) curve 914 that illustrates the percentage of water converted to hydrate which is determined as described above. The gas composition chart 908 includes gas component curves 916, 918, 920, 922, 924, 926, 928 representing mole percentages of various gas components that are determined as described above.

In an illustrative embodiment, the total feed composition or gas composition can be managed in “Fluid Data Management” interface similar to the interface 802 shown in FIG. 8. The pressure-temperature data as a function of time obtained from the laboratory experiment can be uploaded or entered manually in a “Closed System Hydrate Modeling” interface. The interface also may allow for adjustment of the total amount of gas and water in terms of mass or volume if the user does not know the total feed composition (i.e., when all phases, such as water and gas, are mixed in the autoclave cell). Hence, the computing device may perform an automated calculation of the overall feed composition. This needs adjusting the fluid composition by eliminating the water fraction if it presents. Subsequently, the feed composition is updated by considering the quantities of water and gas, along with the composition of free-water gas.

The interface may provide the option for the user to select different hydrate structures (e.g., Structure I and Structure II) and instruct it to specifically calculate the chosen structure. However, the structure optimization through the Gibbs minimization algorithm can be selected to allow the software to predict the most stable hydrate structure depending on the composition and pressure-temperature data, as described above. Accordingly, this feature enables the prediction of not only a single stable hydrate structure but also the simultaneous formation of multiple stable hydrate structures under specific pressure-temperature conditions.

Once all data are provided to the system, the hydrate fraction can be calculated by running the “CPA-SRK with Gibbs minimization” model which implements the kinetic hydrate flash algorithm as described above. The pressure, temperature, and the calculated hydrate fraction formed in the laboratory experiment can be displayed, for example using the interface 902. In some embodiments, a data file such as Excel report file may be generated by the system. This data may include all details of kinetic hydrate flash calculation such as hydrate structure and composition of phases (e.g., gas, aqueous, liquid hydrocarbon, and hydrate).

In an illustrative experiment, the system 100 was used with laboratory experimental data to analyze the hydrate fraction and gas composition during the hydrate formation process in an autoclave cell 104. The test conditions for the experiment are described in Table 1, below. A synthetic natural gas with the composition listed in Table 2, below, was used.

TABLE 1
Test conditions.

| Initial | Initial | Water | Gas | Test |
|---|---|---|---|---|
| Pressure | Temperature | Volume | Volume | Temperature |
| bar | ° C. | cc | cc | ° C. |
| 80 | 24 | 220 | 220 | 1 |

TABLE 2
Composition of synthetic natural gas.

| Component | Mole % |
|---|---|
| Methane (C₁) | 79.68 |
| Ethane (C₂) | 10.84 |
| Propane (C₃) | 4.63 |
| i-Butane (i-C₄) | 0.62 |
| n-Butane (n-C₄) | 1.12 |
| Carbon dioxide (CO₂) | 1.36 |
| Nitrogen (N₂) | 1.75 |

In this experiment, the autoclave 104 was cooled down to test temperature (1° C.) without mixing. This lack of mixing prevents hydrate formation. Once the system reached 1° C., the mixing was initiated to induce hydrate formation. Pressure-temperature profile as a function of time is shown in the chart 904 of the interface 902 in FIG. 9. The formation of gas hydrates is indicated by a pressure drop and often a corresponding increase in temperature. The decrease in pressure is a consequence of gas being incorporated into the hydrate crystal structure. The increase in temperature is observed because gas hydrate formation is an exothermic reaction.

The hydrate fraction (WCH %: the percentage of water converted to hydrate) as a function of time predicted by the system 100 is shown in the chart 906 of FIG. 9. As shown, the system 100 predicts that no hydrate formation occurred during the cool-down phase when there was no mixing in the system. This suggests that the pressure drop during the cool-down phase was solely attributed to gas compression due to the decreasing temperature, as predicted by the software. However, once the mixing was started the hydrate fraction was increased as predicted by the system 100. The system 100 predicts 40% of water was converted to hydrate at the end of experiment.

Additionally, as described above, the system 100 implemented algorithm enables the prediction of gas composition during the hydrate formation process, alongside the hydrate fraction profile as a function of time. The gas composition profile during hydrate formation may be used as a validation for the developed algorithm described above as well.

The chart 908 of FIG. 9 shows the predicted mole % of gas components in the autoclave cell 104 during the hydrate formation process for the aforementioned experiment. The results show that the gas composition remains constant during the cool-down phase where hydrate formation did not occur due to the absence of mixing. However, once the mixing started and initiated hydrate formation, the gas composition underwent changes. As shown, the system 100 predicts that during the process, the mole % of the most thermodynamically stable hydrate formers (e.g., C₃ and C₄ as indicated by the curves 920, 924, 928) decreased, while the mole % of methane (the curve 916) and nitrogen (the curve 922), which are less thermodynamically stable hydrate formers in this particular case, increased. These results align with the gas consumption pattern by the hydrate crystals in order of thermodynamic stability.

Furthermore, in order to validate the multiphase hydrate flash loop as described above, the results of the vapor phase composition and the hydrate mole fraction were compared with the experimental data, the system 100 in this disclosure, two commercial software packages (HydraFlash and PVTsim), and a reference model for the composition listed in Table 3, below. The hydrate mole fraction presented here represents the equilibrium mole fraction. This means it is the maximum hydrate mole fraction that can form in a system under specific temperature, pressure, and composition conditions. This value does not correspond to the kinetic hydrate mole fraction observed in laboratory experiments, which can be calculated using the kinetic hydrate flash algorithm as described herein.

TABLE 3
Composition of the system for hydrate multiphase flash.

| Component | Mole % |
|---|---|
| Methane (C₁) | 87.44 |
| Ethane (C₂) | 6.00 |
| Propane (C₃) | 2.43 |
| i-Butane (i-C₄) | 0.20 |
| n-Butane (n-C₄) | 0.30 |
| Carbon dioxide (CO₂) | 2.13 |
| Nitrogen (N₂) | 1.75 |
| Water/Hydrocarbon (mole) | 6.12 |

As the results in Table 4, below, indicate, the multiphase hydrate flash loop as disclosed herein is capable of predicting equilibrium hydrate mole fraction and phase composition that closely aligns with the experimental data and predictions from commercial software packages. This means the multiphase hydrate flash loop as described herein is proficient in accurately predicting the most stable hydrate composition to minimize the Gibbs free energy.

TABLE 4
Experimental and predicted vapor phase composition and equilibrium hydrate mole fraction
as a results of hydrate multiphase flash for the composition listed in Table 3.

| | | | | | | | | | | | Mean | Hydrate |
|---|---|---|---|---|---|---|---|---|---|---|---|---|
| P | T | | Vapor phase composition | | | | | | | | relative | mole |
| (MPa) | (° C.) | | N₂ | CO₂ | C₁ | C₂ | C₃ | iC₄ | nC₄ | H₂O | error | fraction |
| 6.205 | 287.2 | Experiment | 1.63 | 1.69 | 90.2 | 5.00 | 1.08 | 0.09 | 0.27 | — | — | — |
| | | System 100 | 1.66 | 1.89 | 90.31 | 4.82 | 0.93 | 0.08 | 0.30 | 0.02 | 0.08 | 0.1150 |
| | | HydraFlash | 1.66 | 1.89 | 90.41 | 4.80 | 0.91 | 0.07 | 0.23 | 0.03 | 0.10 | 0.1194 |
| | | PVTsim | 1.70 | 1.81 | 90.99 | 4.48 | 0.71 | 0.05 | 0.23 | 0.03 | 0.17 | 0.1447 |
| | | Reference | 1.64 | 2.00 | 90.00 | 5.00 | 1.03 | 0.08 | 0.25 | 0.04 | 0.06 | 0.1032 |
| | | Model | | | | | | | | | | |

What is claimed is:

1. A method for multiphase hydrate fraction modeling, the method comprising:
receiving, by a computing device, an initial feed composition of a constant volume cell;
receiving, by the computing device, a time series of pressure data and temperature data for the constant volume cell during a test procedure;
determining, by the computing device, a non-equilibrium hydrate fraction for the constant volume cell based on the time series of pressure data and temperature data; and
predicting, by the computing device, one or more phase components of the constant volume cell based on the non-equilibrium hydrate fraction.

2. The method of claim 1, wherein the initial feed composition is indicative of water volume or mass, liquid hydrocarbon volume or mass, gas volume or mass, and gas composition initially included in the constant volume cell.

3. The method of claim 1, wherein the constant volume cell comprises an autoclave.

4. The method of claim 1, wherein the constant volume cell comprises an undersea pipeline.

5. The method of claim 1, wherein determining the non-equilibrium hydrate fraction for the constant volume cell based on the time series of pressure data and temperature data comprises, for each time index of the time series:
determining whether hydrate formation has occurred based on the pressure data and the temperature data;
performing multiphase isothermal hydrate flash by successive substitution to determine a most stable hydrate composition at the time index in response to determining that hydrate formation has occurred; and
updating the non-equilibrium hydrate fraction based on the most stable hydrate composition determined by the multiphase isothermal hydrate flash.

6. The method of claim 5, wherein determining whether hydrate formation has occurred comprises:
determining whether the constant volume cell is in a hydrate stability zone based on the temperature data and the pressure data at the time index;
determining phase molar density of non-hydrate phase at the time index by performing an isothermal pressure-temperature (PT) flash in response to determining that the constant volume cell is in the hydrate stability zone; and
comparing the phase molar density of the non-hydrate phase to a phase density of the non-hydrate phase at a previous time index to determine whether hydrate formation has occurred.

7. The method of claim 5, wherein the most stable hydrate composition is indicative of a hydrate phase and a non-hydrate phase, wherein the hydrate phase is indicative of one or more hydrate structures, and wherein the non-hydrate phase is indicative of a vapor phase or a plurality of liquid phases.

8. The method of claim 5, wherein updating the non-equilibrium hydrate fraction comprises:
estimating an initial hydrate fraction based on a pressure drop at the time index and a hydration number, wherein the hydration number is based on hydrate composition of the most stable hydrate composition;
determining a first molar volume or density of the non-hydrate phase based on the most stable hydrate composition and the initial hydrate fraction;
determining a second molar volume or density of the non-hydrate phase based on a pressure-temperature flash for non-hydrate phase composition of the most stable hydrate composition;
updating the initial hydrate fraction as a function of the first molar volume or density and the second molar volume or density; and
continuing to update the initial hydrate fraction until the first molar volume or density and the second molar volume or density converge to a predetermined percentage error.

9. The method of claim 1, further comprising displaying, by the computing device, the non-equilibrium hydrate fraction over the time series.

10. The method of claim 1, further comprising displaying, by the computing device, gas composition in the constant volume cell over the time series based on predicting the one or more phase components.

11. A computing device for multiphase hydrate fraction modeling, the computing device comprising:
a test interface to (i) receive an initial feed composition of a constant volume cell, and (ii) receive a time series of pressure data and temperature data for the constant volume cell during a test procedure; and
an analytical engine to (i) determine a non-equilibrium hydrate fraction for the constant volume cell based on the time series of pressure data and temperature data, and (ii) predict one or more phase components of the constant volume cell based on the non-equilibrium hydrate fraction.

12. The computing device of claim 11, wherein the initial feed composition is indicative of water volume or mass, liquid hydrocarbon volume or mass, gas volume or mass, and gas composition initially included in the constant volume cell.

13. The computing device of claim 11, wherein the constant volume cell comprises an autoclave.

14. The computing device of claim 11, wherein the constant volume cell comprises an undersea pipeline.

15. The computing device of claim 11, wherein to determine the non-equilibrium hydrate fraction for the constant volume cell based on the time series of pressure data and temperature data comprises, for each time index of the time series, to:
determine whether hydrate formation has occurred based on the pressure data and the temperature data;
perform multiphase isothermal hydrate flash by successive substitution to determine a most stable hydrate composition at the time index in response to a determination that hydrate formation has occurred; and
update the non-equilibrium hydrate fraction based on the most stable hydrate composition determined by the multiphase isothermal hydrate flash.

16. The computing device of claim 15, wherein to determine whether hydrate formation has occurred comprises to:
determine whether the constant volume cell is in a hydrate stability zone based on the temperature data and the pressure data at the time index;
determine phase molar density of non-hydrate phase at the time index by performance of an isothermal pressure-temperature (PT) flash in response to a determination that the constant volume cell is in the hydrate stability zone; and
compare the phase molar density of the non-hydrate phase to a phase density of the non-hydrate phase at a previous time index to determine whether hydrate formation has occurred.

17. The computing device of claim 15, wherein the most stable hydrate composition is indicative of a hydrate phase and a non-hydrate phase, wherein the hydrate phase is indicative of one or more hydrate structures, and wherein the non-hydrate phase is indicative of a vapor phase or a plurality of liquid phases.

18. The computing device of claim 15, wherein to update the non-equilibrium hydrate fraction comprises to:
estimate an initial hydrate fraction based on a pressure drop at the time index and a hydration number, wherein the hydration number is based on hydrate composition of the most stable hydrate composition;
determine a first molar volume or density of the non-hydrate phase based on the most stable hydrate composition and the initial hydrate fraction;
determine a second molar volume or density of the non-hydrate phase based on a pressure-temperature flash for non-hydrate phase composition of the most stable hydrate composition;
update the initial hydrate fraction as a function of the first molar volume or density and the second molar volume or density; and
continue to update the initial hydrate fraction until the first molar volume or density and the second molar volume or density converge to a predetermined percentage error.

19. The computing device of claim 11, further comprising a user interface to display the non-equilibrium hydrate fraction over the time series.

20. The computing device of claim 11, further comprising a user interface to display gas composition in the constant volume cell over the time series based on a prediction of the one or more phase components.
